(19) United States
(12) Patent Application Publication
(10) Pub. No.: US 2011/0020448 A1
(43) Pub. Date: Jan. 27, 2011

(54) PHARMACEUTICAL COMPOSITION FOR THE TREATMENT AND PREVENTION OF GLAUCOMA

(75) Inventors: Myung-Gyu Park, Gyeonggi-do (KR); Taehwan Kwak, Gyeonggi-do (KR)

Correspondence Address: LADAS & PARRY, 5670 WILSHIRE BOULEVARD, SUITE 2100, LOS ANGELES, CA, 90036-5679, US

(73) Assignees: MAZENCE, INC., DAEJEON (KR); KT & G CORPORATION, DAEJEON (KR)

(21) Appl. No.: 12/747,867

(22) PCT Filed: Dec. 18, 2008
(86) PCT No.: PCT/KR08/07507

§ 371 Date: Sep. 27, 2010

(30) Foreign Application Priority Data
Dec. 24, 2007 (KR) 10-2007-0136105

(51) Int. Cl. A61K 31/352; C07D 311/92; C07D 307/92; A61K 31/343; C07D 307/94; A61K 9/00; A61P 27/06

(52) U.S. Cl. 424/484; 549/389; 514/454; 549/385; 514/468; 549/395; 549/331

(57) ABSTRACT

Provided is a pharmaceutical composition for the treatment and prevention of glaucoma, containing (a) a therapeutically effective amount of a compound represented by Formula 1 or a pharmaceutically acceptable salt, prodrug, solvate or isomer thereof, and (b) a pharmaceutically acceptable carrier, diluent or excipient or any combination thereof.

### FIELD OF THE INVENTION

The present invention relates to a pharmaceutical composition for the treatment and prevention of glaucoma. More specifically, the present invention relates to a pharmaceutical composition having excellent effects for the treatment and prevention of glaucoma, containing (a) a therapeutically effective amount of a naphthoquinone-based compound or a pharmaceutically acceptable salt, prodrug, solvate or isomer thereof as an active ingredient and (b) a pharmaceutically acceptable carrier, diluent or excipient or any combination thereof.

### BACKGROUND OF THE INVENTION

The outer surface of the human eye serves as lens to focus incoming light onto the retina located at the rear of the eyeball, and the retina receives the light and transmits a variety of visual information to the brain by way of the optic nerve. Glaucoma is the medical condition which is accompanied by visual disorders due to damage of the optic nerve responsible for transmission of information from the eyes to the brain.

Glaucoma is a disease that takes place due to optic nerve injury or damage resulting in no communication of information, so a variety of factors that may impair the optic nerve can contribute to the pathogenesis of glaucoma. Since pathogenic mechanisms, pathogenic causes and symptoms of glaucoma are extensively diverse as described above, glaucoma is regarded as single disease entities as well as multiple disease entities.

Common symptoms of glaucoma may include, for example, elevation of intraocular pressure (IOP), glaucomatous optic disc cupping and subsequent abnormal visual defect. Damage of the eye structure and function due to glaucoma may result in loss of one's eyesight. Further, when internal pressure of the eye which is dependent on an amount of aqueous humor present in the eye, that is, the intraocular pressure is abnormally high due to glaucoma, the eye becomes hard, which may lead to dysfunction of the retinal nerve fiber and the optic nerve. This may result in death of the optic nerve, and the once-dead optic nerve cannot revive unlike other ophthalmic diseases, thus causing narrowing of the visual field and finally permanent blindness.

Glaucoma may be broadly classified into three types: congenital (developmental) glaucoma, primary glaucoma with unclear causes, and secondary glaucoma which is caused by ocular trauma or drug side effects. Glaucoma generally refers to primary glaucoma.

Patients with congenital glaucoma are born with maldevelopment of the anterior chamber angle, and obstruction of the aqueous outflow causes this type of glaucoma. Primary glaucoma is further subdivided into two types with manifestation of different symptoms, open-angle glaucoma and angle-closure glaucoma, depending on the blockage of the anterior chamber angle where aqueous humor flows out of the eye.

Open-angle glaucoma is a type of glaucoma which is accompanied by the elevation of intraocular pressure arising as a result of malfunction of the aqueous outflow system due to increased resistance of the trabecular meshwork through which aqueous humor flows although the anterior chamber angle is open. Angle-closure glaucoma takes place with clinical symptoms of elevated intraocular pressure resulting from blockage of the aqueous outflow due to obstruction of the anterior chamber angle. Acute angle-closure glaucoma is an episode with sudden blockage of the anterior chamber angle. In this case, the intraocular pressure rapidly rises to cause severe pain of the eyes, headache, nauseation, and amblyopia.

Secondary glaucoma may be caused by various pathogenic factors such as ocular trauma, inflammations, tumors, long-standing cataracts and diabetes. Secondary glaucoma may also result from long-term use of steroid drugs for the treatment of other diseases. Application of steroids may lead to the elevation of intraocular pressure, thus causing glaucoma.

For the treatment of glaucoma, laser treatment, surgical therapy, or the like is performed when IOP cannot be controlled with a drug, but drug therapy is used as the first line therapy.

Drugs conventionally used in the drug therapy of glaucoma include sympathetic nerve stimulants (such as epinephrine, apraclonidine, etc.), sympathetic nerve blockers (such as timolol, befunolol, carteolol, nipradilol, betaxolol, levobunolol, metipranolol, etc.), parasympathetic nerve agonists (such as pilocarpine, etc.), carbonic anhydrase inhibitors (such as acetazolamide, etc.), prostaglandins (such as isopropyl unoprostone, latanoprost, travoprost, bimatoprost, etc.), and so forth.

However, most of these therapeutic agents are eye drops which merely exhibit intraocular pressure-lowering effects and are reported to show various drug side effects such as eye burning sensation upon instillation of drugs in the eyes, and ocular discoloration upon chronic administration of drugs. Accordingly, there is an urgent need for development of active agents as safe anti-glaucoma medications which are capable of reducing side effects.

To this end, the inventors of the present invention have discovered that certain naphthoquinone compounds can exhibit excellent prophylactic and therapeutic effects against glaucoma.

Meanwhile, some of pharmaceutical compositions containing conventional naphthoquinone-based compounds as an active ingredient are known in the art. Of these naphthoquinone-based compounds, β-lapachone is derived from the laphacho tree (Tabebuia avellanedae) which is native to South America, and dunnione and α-dunnione are also derived from the leaves of Streptocarpus dunnii native to South America. These naturally-occurring tricyclic naphthoquinone derivatives have been used for a long time, not only as anti-cancer medications, but also as medications for the treatment of a Chagas disease known as a representative endemic disease of South America, and were also known to exhibit potent efficacies. In particular, pharmacological actions of these naphthoquinone derivatives as anticancer medications have drawn a great deal of attention since they were known to the Western nations. As disclosed in U.S. Pat. No. 5,969,163, a number of anti-cancer drugs employing the tricyclic naphthoquinone derivatives are being actually developed by many research groups.

Despite the various researches carried out in the related area, there is no report demonstrating that these naphthoquinone-based compounds exhibit pharmacologically beneficial effects on the treatment or prevention of glaucoma.

### SUMMARY OF THE INVENTION

As a result of a variety of extensive and intensive studies and experiments to solve the problems as described above, the inventors of the present invention have newly demonstrated that certain naphthoquinone-based compounds can be used for the treatment or prevention of glaucoma, and have discovered that these compounds can exert desired pharmacological effects, when formulated to be absorbable into target sites of the body. The present invention has been completed based on these findings.

In accordance with an aspect of the present invention, the above and other objects can be accomplished by the provision of a pharmaceutical composition for the treatment and prevention of glaucoma, comprising: (a) a therapeutically effective amount of a compound represented by Formula 1 below: or a pharmaceutically acceptable salt, prodrug, solvate or isomer thereof; and (b) a pharmaceutically acceptable carrier, diluent or excipient or any combination thereof.

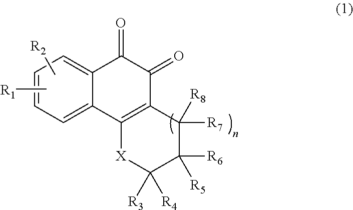

wherein:

R₁ and R₂ are each independently hydrogen, halogen, hydroxyl, or C₁-C₆ lower alkyl or alkoxy, or R₁ and R₂ may be taken together to form a cyclic structure which may be saturated or partially or completely unsaturated;

R₃, R₄, R₅, R₆, R₇ and R₈ are each independently hydrogen, hydroxyl, C₁-C₂₀ alkyl, alkene or alkoxy, cycloalkyl, heterocycloalkyl, aryl or heteroaryl, or two of R₃ to R₈ may be taken together to form a cyclic structure which may be saturated or partially or completely unsaturated;

X is selected from the group consisting of C(R)(R′), N(R″) wherein R, R′ and R″ are each independently hydrogen or C₁-C₆ lower alkyl, O and S, preferably O or S, and more preferably O; and

n is 0 or 1, with proviso that when n is 0, carbon atoms adjacent to n form a cyclic structure via a direct bond

According to the experiments conducted by the inventors of the present invention, it was observed that glaucoma-induced rats are susceptible to oxidative stress. Such oxidative stress is believed to be involved in the onset of glaucoma, upon considering that the oxidative stress accelerates the optic nerve damage or injury causing glaucoma while increasing the production of toxic reactive oxygen species, and causes degeneration of retinal ganglion cells (RGCs) and RGC axons forming the optic nerve.

As a result of repeated extensive and intensive studies and experiments based on the facts described above, the inventors of the present invention have confirmed that the aforementioned naphthoquinone-based compounds exhibit excellent effects on the prevention and treatment of glaucoma. This is believed to be due to that the naphthoquinone-based compounds of the present invention reduce reactive oxygen species-induced oxidative damage to thereby prevent degeneration of RGCs and RGC axons.

As used herein, the term “pharmaceutically acceptable salt” means a formulation of a compound that does not cause significant irritation to an organism to which it is administered and does not abrogate the biological activity and properties of the compound. Examples of the pharmaceutical salt may include acid addition salts of the compound with acids capable of forming a non-toxic acid addition salt containing pharmaceutically acceptable anions, for example, inorganic acids such as hydrochloric acid, sulfuric acid, nitric acid, phosphoric acid, hydrobromic acid and hydroiodic acid; organic carbonic acids such as tartaric acid, formic acid, citric acid, acetic acid, trichloroacetic acid, trifluoroacetic acid, gluconic acid, benzoic acid, lactic acid, fumaric acid, maleic acid and salicylic acid; or sulfonic acids such as methanesulfonic acid, ethanesulfonic acid, benzenesulfonic acid and p-toluenesulfonic acid. Specifically, examples of pharmaceutically acceptable carboxylic acid salts include salts with alkali metals or alkaline earth metals such as lithium, sodium, potassium, calcium and magnesium, salts with amino acids such as arginine, lysine and guanidine, salts with organic bases such as dicyclohexylamine, N-methyl-D-glucamine, tris(hydroxymethyl)methylamine, diethanolamine, choline and triethylamine. The compound of the Formula 1 or 2 in accordance with the present invention may be converted into salts thereof, by conventional methods well-known in the art.

As used herein, the term “prodrug” means an agent that is converted into the parent drug in vivo. Prodrugs are often useful because, in some situations, they may be easier to administer than the parent drug. They may, for instance, be bioavailable by oral administration, whereas the parent may be not. The prodrugs may also have improved solubility in pharmaceutical compositions over the parent drug. An example of a prodrug, without limitation, would be a compound of the present invention which is administered as an ester (the “prodrug”) to facilitate transport across a cell membrane where water-solubility is detrimental to mobility, but which then is metabolically hydrolyzed to the carboxylic acid, the active entity, once inside the cell where water solubility is beneficial. A further example of the prodrug might be a short peptide (polyamino acid) bonded to an acidic group, where the peptide is metabolized to reveal the active moiety.

As an example of such prodrug, the pharmaceutical compounds in accordance with the present invention can include a prodrug represented by Formula 1 a below as an active material:

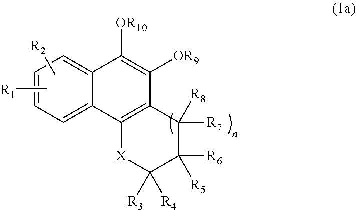

wherein,

R₁, R₂, R₃, R₄, R₅, R₆, R₇, R₈, X and n are as defined in Formula 1;

R₉ and R₁₀ are each independently —SO₃⁻Na⁺ or substituent represented by Formula A below or a salt thereof,

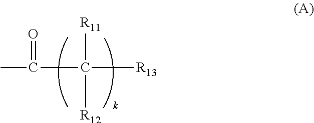

wherein,

R₁₁ and R₁₂ are each independently hydrogen or substituted or unsubstituted C₁-C₂₀ linear alkyl or C₁-C₂₀ branched alkyl

R₁₃ is selected from the group consisting of substituents i) to viii) below:

i) hydrogen;

ii) substituted or unsubstituted C₁-C₂₀ linear alkyl or C₁-C₂₀ branched alkyl;

iii) substituted or unsubstituted amine;

iv) substituted or unsubstituted C₃-C₁₀ cycloalkyl or C₃-C₁₀ heterocycloalkyl;

v) substituted or unsubstituted C₄-C₁₀ aryl or C₄-C₁₀ heteroaryl;

vi) —(CRR′—NR″CO)₁-R₁₄, wherein R, R′ and R″ are each independently hydrogen or substituted or unsubstituted C₁-C₂₀ linear alkyl or C₁-C₂₀ branched alkyl, R₁₄ is selected from the group consisting of hydrogen, substituted or unsubstituted amine, cycloalkyl, heterocycloalkyl, aryl and heteroaryl, 1 is selected from the 1˜5;

vii) substituted or unsubstituted carboxyl;

viii) —OSO₃⁻Na⁺;

k is selected from the 0˜20, with proviso that when k is 0, R₁₁ and R₁₂ are not anything, and R₁₃ is directly bond to a carbonyl group.

As used herein, the term “solvate” means a compound of the present invention or a salt thereof, which further includes a stoichiometric or non-stoichiometric amount of a solvent bound thereto by non-covalent intermolecular forces. Preferred solvents are volatile, non-toxic, and/or acceptable for administration to humans. Where the solvent is water, the solvate refers to a hydrate.

As used herein, the term “isomer” means a compound of the present invention or a salt thereof, that has the same chemical formula or molecular formula but is optically or sterically different therefrom. Unless otherwise specified, the term “compound of Formula 1 or Formula 2” is intended to encompass a compound per se, and a pharmaceutically acceptable salt, prodrug, solvate and isomer thereof.

As used herein, the term “alkyl” refers to an aliphatic hydrocarbon group. The alkyl moiety may be a “saturated alkyl” group, which means that it does not contain any alkene or alkyne moieties. Alternatively, the alkyl moiety may also be an “unsaturated alkyl” moiety, which means that it contains at least one alkene or alkyne moiety. The term “alkene” moiety refers to a group in which at least two carbon atoms form at least one carbon-carbon double bond, and an “alkyne” moiety refers to a group in which at least two carbon atoms form at least one carbon-carbon triple bond. The alkyl moiety, regardless of whether it is substituted or unsubstituted, may be branched, linear or cyclic.

As used herein, the term “heterocycloalkyl” means a carbocyclic group in which one or more ring carbon atoms are substituted with oxygen, nitrogen or sulfur and which includes, for example, but is not limited to furan, thiophene, pyrrole, pyrroline, pyrrolidine, oxazole, thiazole, imidazole, imidazoline, imidazolidine, pyrazole, pyrazoline, pyrazolidine, isothiazole, triazole, thiadiazole, pyran, pyridine, piperidine, morpholine, thiomorpholine, pyridazine, pyrimidine, pyrazine, piperazine and triazine.

As used herein, the term “aryl” refers to an aromatic substituent group which has at least one ring having a conjugated pi (π) electron system and includes both carbocyclic aryl (for example, phenyl) and heterocyclic aryl (for example, pyridine) groups. This term includes monocyclic or fused-ring polycyclic (i.e., rings which share adjacent pairs of carbon atoms) groups.

As used herein, the term “heteroaryl” refers to an aromatic group that contains at least one heterocyclic ring.

Examples of aryl or heteroaryl include, but are not limited to, phenyl, furan, pyran, pyridyl, pyrimidyl and triazyl.

R₁, R₂, R₃, R₄, R₅, R₆, R₇ and R₈ in Formula 1 or Formula 2 in accordance with the present invention may be optionally substituted. When substituted, the substituent group(s) is(are) one or more group(s) individually and independently selected from cycloalkyl, aryl, heteroaryl, heteroalicyclic, hydroxy, alkoxy, aryloxy, mercapto, alkylthio, arylthio, cyano, halogen, carbonyl, thiocarbonyl, O-carbamyl, N carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino including mono and di substituted amino, and protected derivatives thereof

Among compounds of Formula 1, preferred are compounds of Formulas 3 and 5 below.

Compounds of Formula 3 are compounds wherein n is 0 and adjacent carbon atoms form a cyclic structure (furan ring) via a direct bond therebetween and are often referred to as “furan compounds” or “furano-o-naphthoquinone derivatives” hereinafter.

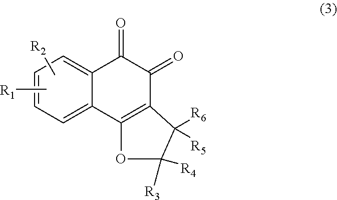

Compounds of Formula 4 are compounds wherein n is 1 and are often referred to as “pyran compounds” or “pyrano-o-naphthoquinone” hereinafter.

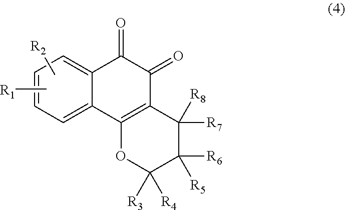

In Formula 1, each of R₁ and R₂ is particularly preferably hydrogen.

Among the furan compounds of Formula 3, particularly preferred are compounds of Formula 3a wherein R₁, R₂ and R₄ are hydrogen, or compounds of Formula 3b wherein R₁, R₂ and R₆ are hydrogen.

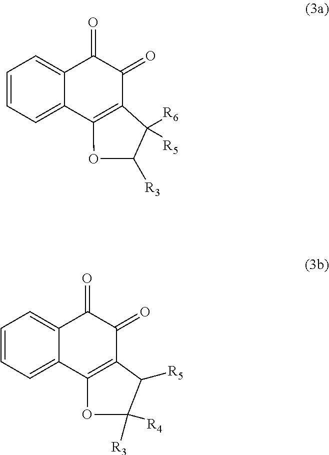

Further, among the pyran compounds of Formula 4, particularly preferred are compounds of Formula 4a wherein R₁, R₂, R₅, R₆, R₇ and R₈ are respectively hydrogen,

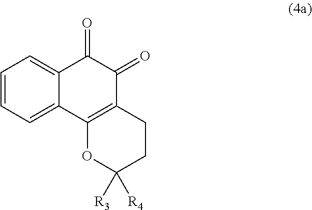

The term “pharmaceutical composition” as used herein means a mixture of a compound of Formula 1 or Formula 2 with other chemical components, such as diluents or carriers. The pharmaceutical composition facilitates administration of the compound to an organism. Various techniques of administering a compound are known in the art and include, but are not limited to oral, injection, aerosol, parenteral and topical administrations. Pharmaceutical compositions can also be obtained by reacting compounds of interest with acids such as hydrochloric acid, hydrobromic acid, sulfuric acid, nitric acid, phosphoric acid, methanesulfonic acid, p-toluenesulfonic acid, salicylic acid and the like.

The term “therapeutically effective amount” means an amount of an active ingredient that is effective to relieve or reduce to some extent one or more of the symptoms of the disease in need of treatment, or to retard initiation of clinical markers or symptoms of a disease in need of prevention, when the compound is administered. Thus, a therapeutically effective amount refers to an amount of the active ingredient which exhibit effects of (i) reversing the rate of progress of a disease; (ii) inhibiting to some extent further progress of the disease; and/or, (iii) relieving to some extent (or, preferably, eliminating) one or more symptoms associated with the disease. The therapeutically effective amount may be empirically determined by experimenting with the compounds concerned in known in vivo and in vitro model systems for a disease in need of treatment.

In the pharmaceutical composition in accordance with the present invention, compounds of Formula 1 or Formula 2 which are active materials, as will be illustrated hereinafter, can be prepared by conventional methods known in the art and/or various processes which are based upon the general technologies and practices in the organic chemistry synthesis field. The preparation processes described below are only exemplary ones and other processes can also be employed. As such, the scope of the instant invention is not limited to the following processes.

### Preparation Method 1: Synthesis of Active Materials by Acid-Catalyzed Cyclization

Tricyclic naphthoquinone (pyrano-o-naphthoquinone and furano-o-naphthoquinone) derivatives having a relatively simple chemical structure are generally synthesized in a relatively high yield via cyclization using sulfuric acid as a catalyst, Based on this process, a variety of compounds of Formula 1 can be synthesized.

More specifically, the above synthesis process may be summarized as follows.

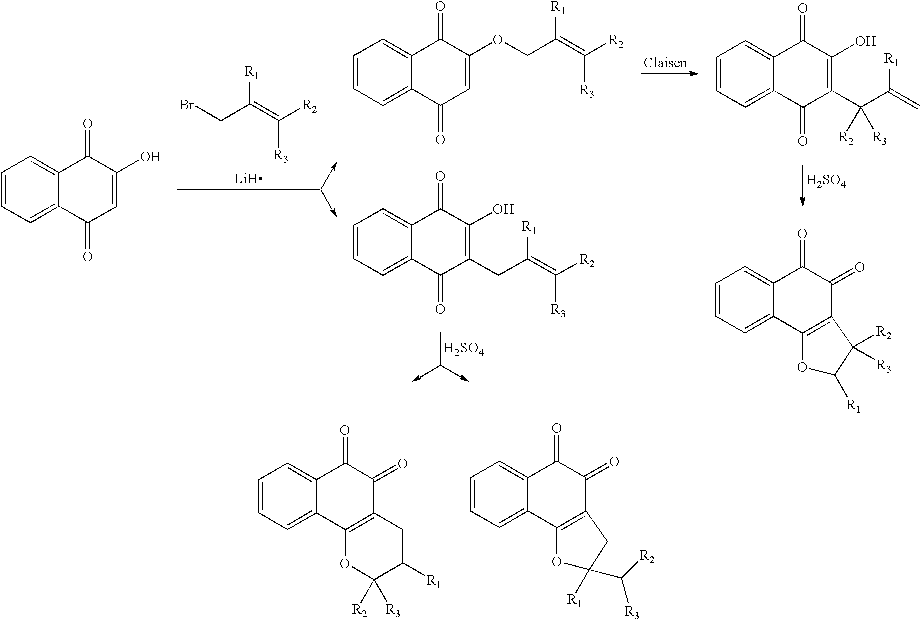

That is, when 2-hydroxy-1,4-naphthoquinone is reacted with various allylic bromides or equivalents thereof in the presence of a base, a C-alkylation product and an O-alkylation product are concurrently obtained. It is also possible to synthesize either of two derivatives only depending upon reaction conditions. Since O-alkylated derivative is converted into another type of C-alkylated derivative through Claisen Rearrangement by refluxing the O-alkylated derivative using a solvent such as toluene or xylene, it is possible to obtain various types of 3-substituted-2-hydroxy-1,4-naphthoquinone derivatives. The various types of C-alkylated derivatives thus obtained may be subjected to cyclization using sulfuric acid as a catalyst, thereby being capable of synthesizing pyrano-o-naphthoquinone or furano-o-naphthoquinone derivatives among compounds of Formula 1.

Preparation Method 2: Diels-Alder Reaction Using 3-methylene-1,2,4-[3H]naphthalenetrione

As taught by V. Nair et al, Tetrahedron Lett. 42 (2001), 4549-4551, it is reported that a variety of pyrano-o-naphthoquinone derivatives can be relatively easily synthesized by subjecting 3-methylene-1,2,4-[3H]naphthalenetrione, produced upon heating 2-hydroxy-1,4-naphthoquinone and formaldehyde together, to Diels-Alder reaction with various olefin compounds. This method is advantageous in that various forms of pyrano-o-naphtho-quinone derivatives can be synthesized in a relatively simplified manner, as compared to induction of cyclization using sulfuric acid as a catalyst.

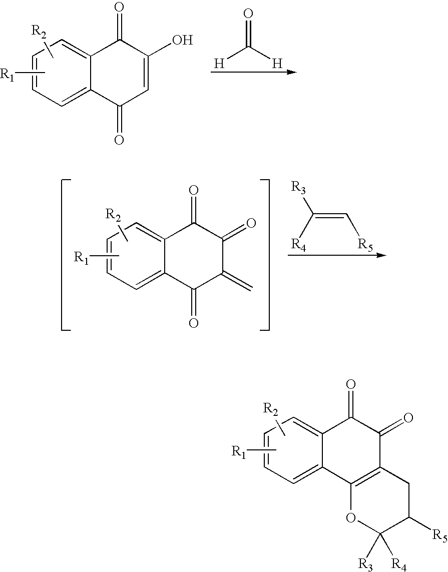

### Preparation Method 3: Haloakylation and Cyclization by Radical Reaction

The same method used in synthesis of Cryptotanshinone and 15,16-dihydro-tanshinone can also be conveniently employed for synthesis of furano-o-naphthoquinone derivatives. That is, as taught by A. C. Baillie et al (J. Chem. Soc. (C) 1968, 48-52), 2-haloethyl or 3-haloethyl radical chemical species, derived from 3-halopropanoic acid or 4-halobutanoic acid derivative, can be reacted with 2-hydroxy-1,4-naphthoquinone to thereby synthesize 3-(2-haloethyl or 3-halopropyl)-2-hydroxy-1,4-naphthoquinone which is then subjected to cyclization under suitable acidic catalyst conditions to synthesize various pyrano-o-naphthoquinone or furano-o-naphthoquinone derivatives.

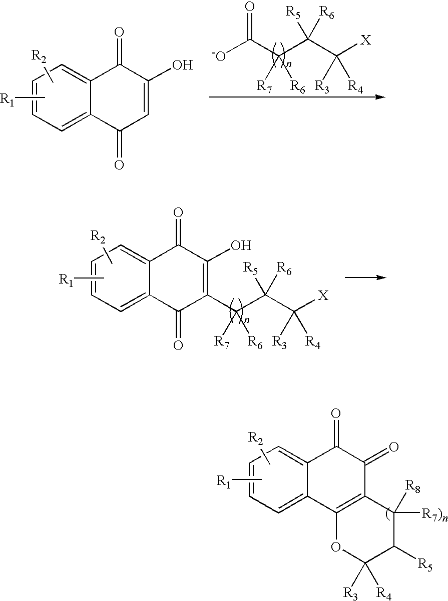

Preparation Method 4: Cyclization of 4,5-benzofurandione by Diels-Alder Reaction

Another method used in synthesis of Cryptotanshinone and 15,16-dihydro-tanshinone may be a method taught by J. K. Snyder et al (Tetrahedron Letters 28 (1987), 3427-3430). According to this method, furano-o-naphthoquinone derivatives can be synthesized by cycloaddition via Diels-Alder reaction between 4,5-benzofurandione derivatives and various diene derivatives.

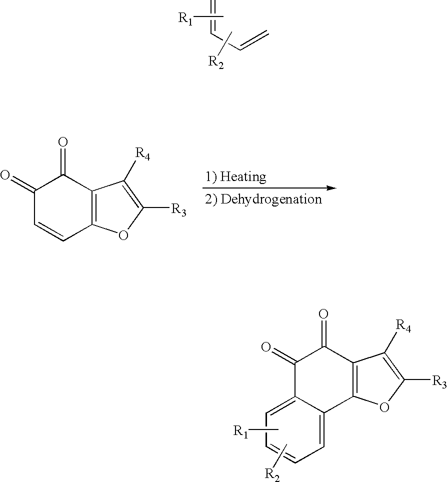

In addition, based on the above-mentioned preparation methods, various derivatives may be synthesized using relevant synthesis methods, depending upon kinds of substituents. Specific examples of derivatives thus synthesized and methods are exemplified in Table 1 below. Specific preparation methods will be described in the following Example.

TABLE 1

| | | | | |
|---|---|---|---|---|
| 1 | 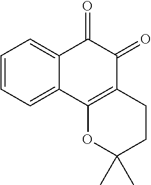 | C₁₅H₁₄O₃ | 242.27 | Method 1 |
| 2 | 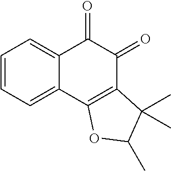 | C₁₅H₁₄O₃ | 242.27 | Method 1 |
| 3 | 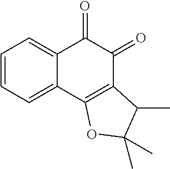 | C₁₅H₁₄O₃ | 242.27 | Method 1 |
| 4 | 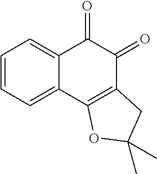 | C₁₄H₁₂O₃ | 228.24 | Method 1 |
| 5 | 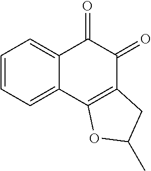 | C₁₃H₁₀O₃ | 214.22 | Method 1 |
| 6 | 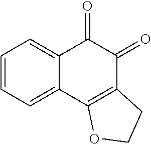 | C₁₂H₈O₃ | 200.19 | Method 2 |
| 7 | 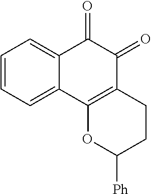 | C₁₉H₁₄O₃ | 290.31 | Method 1 |
| 8 | 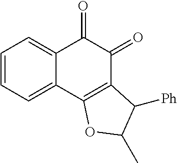 | C₁₉H₁₄O₃ | 290.31 | Method 1 |
| 9 | 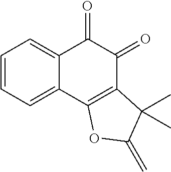 | C₁₅H₁₂O₃ | 240.25 | Method 1 |
| 10 | 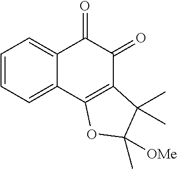 | C₁₆H₁₆O₄ | 272.30 | Method 1 |
| 11 | 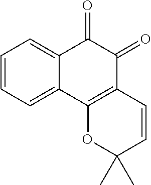 | C₁₅H₁₂O₃ | 240.25 | Method 1 |
| 12 | 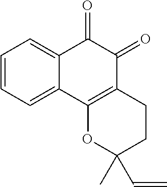 | C₁₆H₁₄O₃ | 254.28 | Method 2 |
| 13 | 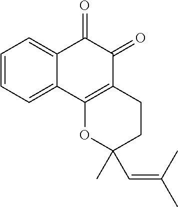 | C₁₈H₁₈O₃ | 282.33 | Method 2 |
| 14 | 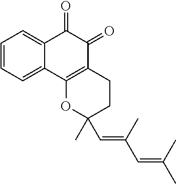 | C₂₁H₂₂O₃ | 322.40 | Method 2 |
| 15 | 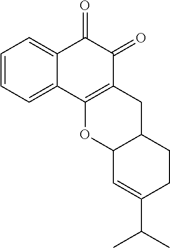 | C₂₁H₂₂O₃ | 322.40 | Method 2 |
| 16 | 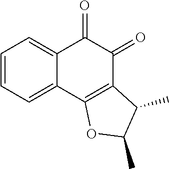 | C₁₄H₁₂O₃ | 228.24 | Method 1 |
| 17 | 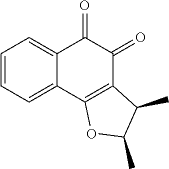 | C₁₄H₁₂O₃ | 228.24 | Method 1 |
| 18 | 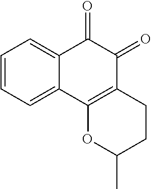 | C₁₄H₁₂O₃ | 228.24 | Method 1 |
| 19 | 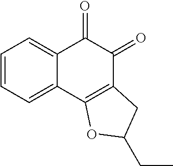 | C₁₄H₁₂O₃ | 228.24 | Method 1 |
| 20 | 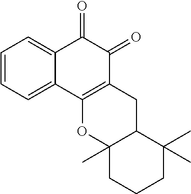 | C₂₀H₂₂O₃ | 310.39 | Method 1 |
| 21 | 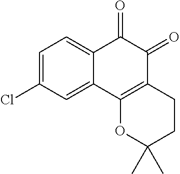 | C₁₅H₁₃ClO₃ | 276.71 | Method 1 |
| 22 | 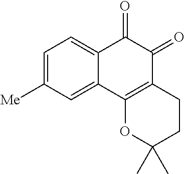 | C₁₆H₁₆O₃ | 256.30 | Method 1 |
| 23 | 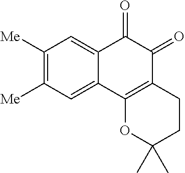 | C₁₇H₁₈O₅ | 302.32 | Method 1 |
| 24 | 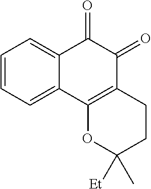 | C₁₆H₁₆O₃ | 256.30 | Method 1 |
| 25 | 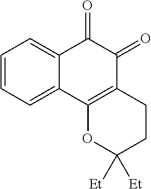 | C₁₇H₁₈O₃ | 270.32 | Method 1 |
| 26 | 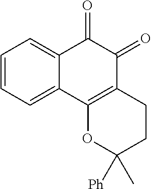 | C₂₀H₁₆O₃ | 304.34 | Method 1 |
| 27 | 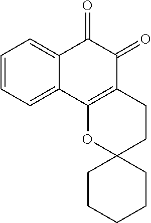 | C₁₈H₁₈O₃ | 282.33 | Method 1 |
| 28 | 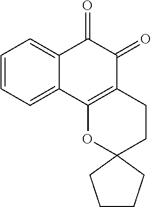 | C₁₇H₁₆O₃ | 268.31 | Method 1 |
| 29 | 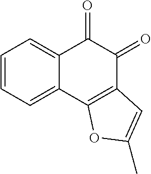 | C₁₃H₈O₃ | 212.20 | Method 1 |
| 30 | 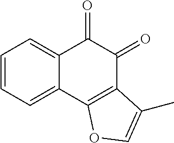 | C₁₃H₈O₃ | 212.20 | Method 4 |
| 31 | 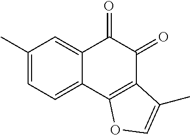 | C₁₄H₁₀O₃ | 226.23 | Method 4 |
| 32 | 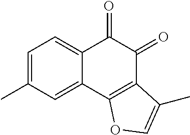 | C₁₄H₁₀O₃ | 226.23 | Method 4 |

The pharmaceutical composition of the present invention may be manufactured in a manner that is itself known, e.g., by means of conventional mixing, dissolving, granulating, dragee-making, levigating, emulsifying, encapsulating, entrapping or lyophilizing processes.

Therefore, pharmaceutical compositions for use in accordance with the present invention may be additionally comprised of a pharmaceutically acceptable carrier, a diluent or an excipient, or any combination thereof. That may be formulated in a conventional manner using one or more pharmaceutically acceptable carriers comprising excipients and auxiliaries which facilitate processing of the active compounds into preparations which can be used pharmaceutically. The pharmaceutical composition facilitates administration of the compound to an organism.

The term “carrier” means a chemical compound that facilitates the incorporation of a compound into cells or tissues. For example, dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffer solution is phosphate buffered saline (PBS) because it mimics the ionic strength conditions of human body fluid. Since buffer salts can control the pH of a solution at low concentrations, a buffer diluent rarely modifies the biological activity of a compound.

The compounds described herein may be administered to a human patient per se, or in the form of pharmaceutical compositions in which they are mixed with other active ingredients, as in combination therapy, or suitable carriers or excipient(s). Proper formulation is dependent upon the route of administration chosen. Techniques for formulation and administration of the compounds may be found in “Remington's Pharmaceutical Sciences,” Mack Publishing Co., Easton, Pa., 18th edition, 1990.

Various techniques relating to pharmaceutical formulation for administering an active ingredient into the body are known in the art and include, but are not limited to oral, injection, aerosol, parenteral and topical administrations. If necessary, they can also be obtained by reacting compounds of interest with acids such as hydrochloric acid, hydrobromic acid, sulfuric acid, nitric acid, phosphoric acid, methanesulfonic acid, p-toluenesulfonic acid, salicylic acid and the like.

Pharmaceutical formulation may be carried out by conventional methods known in the art and, Preferably, the pharmaceutical formulation may be oral, external, transdermal, transmucosal and an injection formulation, and particularly preferred is oral formulation.

The pharmaceutical compounds in accordance with the present invention may be an oral pharmaceutical composition which is prepared into an intestine-targeted formulation. In this connection, the intestine-targeted formulation is not limited to bioabsorption only in the intestine but includes the case where most of the pharmaceutical composition having therapeutic effect is absorbed in the intestine and the remaining may be also absorbed in the organs except the small intestine and the large intestine.

The well-known oral pharmaceutical composition undergoes degradation of active ingredients because many active ingredients are decomposed at oral administration. On the other hand, since the pharmaceutical composition according to the present invention can enhance bioabsorption and bioavailability of an active ingredient via intestine-targeted formulation of the active ingredient.

The intestine-targeted formulation may be designed by taking advantage of numerous physiological parameters of the digestive tract, through a variety of methods. In one preferred embodiment of the present invention, the intestine-targeted formulation may be prepared by (1) a formulation method based on a pH-sensitive polymer, (2) a formulation method based on a biodegradable polymer which is decomposable by an intestine-specific bacterial enzyme, (3) a formulation method based on a biodegradable matrix which is decomposable by an intestine-specific bacterial enzyme, or (4) a formulation method which allows release of a drug after a given lag time, and any combination thereof.

Specifically, the intestine-targeted formulation (1) using the pH-sensitive polymer is a drug delivery system which is based on pH changes of the digestive tract. The pH of the stomach is in a range of 1 to 3, whereas the pH of the small and large intestines has a value of 7 or higher, as compared to that of the stomach. Based on this fact, the pH-sensitive polymer may be used in order to ensure that the pharmaceutical composition reaches the lower intestinal parts without being affected by pH fluctuations of the digestive tract. Examples of the pH-sensitive polymer may include methacrylic acid-ethyl acrylate copolymer (Eudragit: Registered Trademark of Rohm Pharma GmbH).

Preferably, the pH-sensitive polymer may be added by a coating process. For example, addition of the polymer may be carried out by mixing the polymer in a solvent to form an aqueous coating suspension, spraying the resulting coating suspension to form a film coating, and drying the film coating.

The intestine-targeted formulation (2) using the biodegradable polymer which is decomposable by the intestine-specific bacterial enzyme is based on the utilization of a degradative ability of a specific enzyme that can be produced by enteric bacteria. Examples of the specific enzyme may include azoreductase, bacterial hydrolase glycosidase, esterase, polysaccharidase, and the like.

When it is desired to design the intestine-targeted formulation using azoreductase as a target, the biodegradable polymer may be a polymer containing an azoaromatic linkage, for example, a copolymer of styrene and hydroxyethylmethacrylate (HEMA). When the polymer is added to the formulation containing the active ingredient, the active ingredient may be liberated into the intestine by reduction of an azo group of the polymer via the action of the azoreductase which is specifically secreted by enteric bacteria, for example, Bacteroides fragilis and Eubacterium limosum.

When it is desired to design the intestine-targeted formulation using glycosidase, esterase, or polysaccharidase as a target, the biodegradable polymer may be a naturally-occurring polysaccharide or a substituted derivative thereof. For example, the biodegradable polymer may be at least one selected from the group consisting of dextran ester, pectin, amylose, ethyl cellulose and a pharmaceutically acceptable salt thereof. When the polymer is added to the active ingredient, the active ingredient may be liberated into the intestine by hydrolysis of the polymer via the action of each enzyme which is specifically secreted by enteric bacteria, for example, Bifidobacteria and Bacteroides spp. These polymers are natural materials, and have an advantage of low risk of in vivo toxicity.

The intestine-targeted formulation (3) using the biodegradable matrix which is decomposable by an intestine-specific bacterial enzyme may be a form in which the biodegradable polymers are cross-linked to each other and are added to the active ingredient or the active ingredient-containing formulation. Examples of the biodegradable polymer may include naturally-occurring polymers such as chondroitin sulfate, guar gum, chitosan, pectin, and the like. The degree of drug release may vary depending upon the degree of cross-linking of the matrix-constituting polymer.

In addition to the naturally-occurring polymers, the biodegradable matrix may be a synthetic hydrogel based on N-substituted acrylamide. For example, there may be used a hydrogel synthesized by cross-linking of N-tert-butylacryl amide with acrylic acid or copolymerization of 2-hydroxyethyl methacrylate and 4-methacryloyloxyazobenzene, as the matrix. The cross-linking may be, for example an azo linkage as mentioned above, and the formulation may be a form where the density of cross-linking is maintained to provide the optimal conditions for intestinal drug delivery and the linkage is degraded to interact with the intestinal mucous membrane when the drug is delivered to the intestine.

Further, the intestine-targeted formulation (4) with time-course release of the drug after a lag time is a drug delivery system utilizing a mechanism that is allowed to release the active ingredient after a predetermined time irrespective of pH changes. In order to achieve enteric release of the active drug, the formulation should be resistant to the gastric pH environment, and should be in a silent phase for 5 to 6 hours corresponding to a time period taken for delivery of the drug from the body to the intestine, prior to release of the active ingredient into the intestine. The time-specific delayed-release formulation may be prepared by addition of the hydrogel prepared from copolymerization of polyethylene oxide with polyurethane.

Specifically, the delayed-release formulation may have a configuration in which the formulation absorbs water and then swells while it stays within the stomach and the upper digestive tract of the small intestine, upon addition of a hydrogel having the above-mentioned composition after applying the drug to an insoluble polymer, and then migrates to the lower part of the small intestine which is the lower digestive tract and liberates the drug, and the lag time of drug is determined depending upon a length of the hydrogel.

As another example of the polymer, ethyl cellulose (EC) may be used in the delayed-release dosage formulation. EC is an insoluble polymer, and may serve as a factor to delay a drug release time, in response to swelling of a swelling medium due to water penetration or changes in the internal pressure of the intestines due to a peristaltic motion. The lag time may be controlled by the thickness of EC. As an additional example, hydroxypropylmethyl cellulose (HPMC) may also be used as a retarding agent that allows drug release after a given period of time by thickness control of the polymer, and may have a lag time of 5 to 10 hours.

Meanwhile, for injection, the agents of the present invention may be formulated in aqueous solutions, preferably in physiologically compatible buffers such as Hanks's solution, Ringer's solution, or physiological saline. For transmucosal administration, penetrants appropriate to the barrier to be permeated are used in the formulation. Such penetrants are generally known in the art.

The compounds may be formulated for parenteral administration by injection, e.g., by bolus injection or continuous infusion. Formulations for injection may be presented in unit dosage forms, e.g., in ampoules or in multi dose containers, with an added preservative. The compositions may take such forms as suspensions, solutions or emulsions in oily or aqueous vehicles, and may contain formulatory agents such as suspending, stabilizing or dispersing agents.

Alternatively, the active ingredient may be in powder form for constitution with a suitable vehicle, e.g., sterile pyrogen-free water, before use.

Pharmaceutical compositions suitable for use in the present invention include compositions in which the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

When the pharmaceutical composition of the present invention is formulated into a unit dosage form, the compound of Formula 1 or Formula 2 as the active ingredient is preferably contained in a unit dose of about 0.1 to 1,000 mg. The amount of the compound of Formula 1 or Formula 2 administered will be determined by the attending physician, depending upon body weight and age of patients being treated, characteristic nature and the severity of diseases.

In accordance with another aspect of the present invention, there is provided a use of a compound of Formula 1 in the preparation of a medicament for the treatment and prevention of glaucoma. The term “treatment” means ceasing or delaying progress of diseases when the compounds of Formula 1 or compositions comprising the same are administered to subjects exhibiting symptoms of diseases. The term “prevention” means ceasing or delaying symptoms of diseases when the compounds of Formula 1 or compositions comprising the same are administered to subjects exhibiting no symptoms of diseases, but having high risk of developing symptoms of diseases.

### BRIEF DESCRIPTION OF THE DRAWINGS

FIG. 1 is a graph showing a density of the stained retinal ganglion cells as measured in C57BL/6 mouse tissues, under a fluorescence microscope (×400);

FIG. 2 is a graph showing a density of the stained axons as measured in the optic nerve tissue section slide, under a light microscope (×1000); and

FIG. 3 is a graph showing changes in body weight of experimental subject mice as measured after 2-week feeding of animals according to the pair-feeding method.

### DETAILED DESCRIPTION OF PREFERRED EMBODIMENTS

Now, the present invention will be described in more detail with reference to the following Examples. These examples are provided only for illustrating the present invention and should not be construed as limiting the scope and spirit of the present invention.

### Example 1

### Synthesis of β-lapachone (Compound 1)

17.4 g (0.10M) of 2-hydroxy-1,4-naphthoquinone was dissolved in 120 ml of DMSO, and 0.88 g (0.11M) of LiH was gradually added thereto. Here, this should be done with care because hydrogen evolves. The reaction solution was stirred, and after confirming no further production of hydrogen, was additionally stirred for another 30 min. Then, 15.9 g (0.10M) of prenyl bromide (1-bromo-3-methyl-2-butene) and 3.35 g (0.025M) of LiI were gradually added thereto. The reaction solution was heated to 45 and then stirred vigorously for 12 hours at that temperature. The reaction solution was cooled below 10, and 76 g of ice was first added and 250 ml of water was then added. Thereafter, 25 ml of concentrated HCl was gradually added to maintain the resulting solution at an acidic pH>1. 200 ml of EtOAc was added to the reaction mixture which was then stirred vigorously, thereby producing white solids that were not dissolved in EtOAc. These solids were filtered and an EtOAc layer was separated. The aqueous layer was extracted once again with 100 ml of EtOAc and was combined with the previously extracted organic layer. The organic layer was washed with 150 ml of 5% NaHCO₃, and was concentrated. The resulting concentrates were dissolved in 200 ml of CH₂Cl₂, and were vigorously shaken to separate two layers with addition of 70 ml of an aqueous 2N NaOH solution. A CH₂Cl₂ layer was further separated twice with treatment of an aqueous 2N NaOH solution (70 ml×2). The thus-separated aqueous solutions were combined together and adjusted to an acidic pH>2, thereby forming solids. The resulting solids were filtered and separated to give Lapachol. The thus-obtained Lapachol was recrystallized from 75% EtOH. The resulting Lapachol was mixed with 80 ml of sulfuric acid, and the mixture was vigorously stirred at room temperature for 10 min and 200 g of ice was added thereto to complete the reaction. 60 ml of CH₂Cl₂ was added to the reaction materials which were then shaken vigorously. Thereafter, a CH₂Cl₂ layer was separated and washed with 5% NaHCO₃. An aqueous layer was extracted once again using 30 ml of CH₂Cl₂, washed with 5% NaHCO₃ and combined with the previously extracted organic layer. The organic layer was dried over MgSO₄ and concentrated to give impure β-Lapachone. The thus-obtained β-Lapachone was recrystallized from isopropanol, thereby obtaining 8.37 g of pure β-Lapachone.

¹H-NMR (CDCl₃, δ): 8.05 (1H, dd, J=1, 8 Hz), 7.82 (1H, dd, J=1, 8 Hz), 7.64 (1H, dt, J=1, 8 Hz), 7.50 (1H, dt, J=1, 8 Hz), 2.57 (2H, t, J=6.5 Hz), 1.86 (2H, t, J=6.5 Hz) 1.47 (6H, s)

### Example 2

### Synthesis of Dunnione (Compound 2)

In the process of obtaining Lapachol in Example 1, solids separated without being dissolved in EtOAc are 2-prenyloxy-1,4-naphthoquinone, an O-akylation product, unlike Lapachol which is a C-alylation product. The separated 2-prenyloxy-1,4-naphthoquinone was first recrystallized once again from EtOAc. 3.65 g (0.015M) of the thus-purified solids was dissolved in toluene and toluene was refluxed for 5 hours to induce Claisen Rearrangement. Toluene was concentrated by distillation under reduced pressure and was then mixed with 15 ml of sulfuric acid, without further purification. The resulting mixture was stirred vigorously at room temperature for 10 min and 100 g of ice was added thereto to complete the reaction. 50 ml of CH₂Cl₂ was added to the reaction materials which were shaken vigorously. Thereafter, a CH₂Cl₂ layer was separated and washed with 5% NaHCO₃. An aqueous layer was extracted once again using 20 ml of CH₂Cl₂, washed with 5% NaHCO₃ and combined with the previously extracted organic layer. The organic layer was dried over MgSO₄, concentrated and purified by chromatography on silica gel to give 2.32 g of pure Dunnione.

¹H-NMR (CDCl₃, δ): 8.05 (1H, d, J=8 Hz), 7.64 (2H, d, J=8 Hz), 7.56 (1H, m), 4.67 (1H, q, J=7 Hz), 1.47 (3H, d, J=7 Hz), 1.45(3H, s) 1.27 (3H, s)

### Example 3

### Synthesis of α-Dunnione (Compound 3)

4.8 g (0.020M) of 2-prenyloxy-1,4-naphthoquinone purified in Example 2 was dissolved in xylene, and xylene was refluxed for 15 hours, thereby inducing Claisen Rearrangement under significantly higher temperature conditions and prolonged reaction conditions as compared to Example 2. According to this reaction process, α-Dunnione that had progressed to cyclization was obtained together with a Lapachol derivative which had undergone Claisen Rearrangement and in which one of two methyl groups has shifted. Xylene was concentrated by distillation under reduced pressure and purified by chromatography on silica gel to give 1.65 g of pure α-Dunnione.

¹H-NMR (CDCl₃, δ): 8.06 (1H, d, J=8 Hz), 7.64 (2H, m), 7.57 (1H, m), 3.21 (1H, q, J=7 Hz), 1.53 (3H, s), 1.51(3H, s) 1.28 (3H, d, J=7 Hz)

### Example 4

### Synthesis of Compound 4

17.4 g (0.10M) of 2-hydroxy-1,4-naphthoquinone was dissolved in 120 ml of DMSO, and 0.88 g (0.11M) of LiH was gradually added thereto. Here, this should be done with care because hydrogen evolves. The reaction solution was stirred, and after confirming no further production of hydrogen, was additionally stirred for another 30 min. Then, 14.8 g (0.11M) of methallyl bromide (1-bromo-2-methylpropene) and 3.35 g (0.025M) of LiI were gradually added thereto. The reaction solution was heated to 45 and then stirred vigorously for 12 hours at that temperature. The reaction solution was cooled below 10, and 80 g of ice was first added and 250 ml of water was then added. Thereafter, 25 ml of concentrated HCl was gradually added to maintain the resulting solution at an acidic pH>1. 200 ml of CH₂Cl₂ was added to the reaction mixture which was then shaken vigorously to separate two layers. The aqueous layer was extracted once again with addition of 70 ml of CH₂Cl₂ and was combined with the previously extracted organic layer. Two materials were confirmed to be formed newly by TLC and were subsequently used without any particular separation process. The organic layer was concentrated by distillation under reduced pressure, dissolved again in xylene and then refluxed for 8 hours. In this process, two materials on TLC were combined into one, thereby obtaining a relatively pure Lapachol derivative. The thus-obtained Lapachol derivative was mixed with 80 ml of sulfuric acid and stirred vigorously at room temperature for 10 min, and 200 g of ice was added thereto to complete the reaction. 80 ml of CH₂Cl₂ was added to the reaction materials which were then shaken vigorously. Thereafter, a CH₂Cl₂ layer was separated and washed with 5% NaHCO₃. An aqueous layer was extracted once again using 50 ml of CH₂Cl₂, washed with 5% NaHCO₃ and combined with the previously extracted organic layer. The organic layer was dried over MgSO₄ and concentrated to give impure β-Lapachone derivative (Compound 4). The thus-obtained β-Lapachone derivative was recrystallized from isopropanol, thereby obtaining 12.21 g of pure Compound 4.

¹H-NMR (CDCl₃, δ): 8.08 (1H, d, J=8 Hz), 7.64 (2H, m), 7.57 (1H, m), 2.95 (2H, s), 1.61 (6H, s)

### Example 5

### Synthesis of Compound 5

Compound 5 was obtained in the same manner as in Example 4, except that allyl bromide was used instead of methallyl bromide.

¹H-NMR (CDCl₃, δ): 8.07 (1H, d, J=7 Hz), 7.65 (2H, m), 7.58 (1H, m), 5.27 (1H, m), 3.29 (1H, dd, J=10, 15 Hz), 2.75(1H, dd, J=7, 15 Hz), 1.59 (3H, d, J=6 Hz)

### Example 6

### Synthesis of Compound 6

5.08 g (40 mM) of 3-chloropropionyl chloride was dissolved in 20 ml of ether and cooled to −78. 1.95 g (25 mM) of sodium peroxide (Na₂O₂) was gradually added to the resulting solution while being vigorously stirred at that temperature, followed by further vigorous stirring for 30 min. The reaction solution was heated to 0 and 7 g of ice was added thereto, followed by additional stirring for another 10 min. An organic layer was separated, washed once again with 10 ml of cold water at 0, then with an aqueous NaHCO₃ solution at 0. The organic layer was separated, dried over MgSO₄, concentrated by distillation under reduced pressure below 0, thereby preparing 3-chloropropionic peracid.

1.74 g (10 mM) of 2-hydroxy-1,4-naphthoquinone was dissolved in 20 ml of acetic acid, and the previously prepared 3-chloropropionic peracid was gradually added thereto at room temperature. The reaction mixture was refluxed with stirring for 2 hours, and then distilled under reduced pressure to remove acetic acid. The resulting concentrates were dissolved in 20 ml of CH₂Cl₂, and washed with 20 ml of 5% NaHCO₃. An aqueous layer was extracted once again using 20 ml of CH₂Cl₂ and combined with the previously extracted organic layer. The organic layer was dried over MgSO₄ and concentrated to give Compound 6 in admixture with 2-(2-chloroethyl)-3-hydroxy-1,4-naphthoquinone. The resulting mixture was purified by chromatography on silica gel to give 0.172 g of a pure Lapachone derivative (Compound 6).

¹H-NMR (CDCl₃, δ): 8.07 (1H, d, J=7.6 Hz), 7.56˜7.68 (3H, m), 4.89 (2H, t, J=9.2 Hz), 3.17 (2H, t, J=9.2 Hz)

### Example 7

### Synthesis of Compound 7

17.4 g (0.10M) of 2-hydroxy-1,4-naphthoquinone was dissolved in 120 ml of DMSO, and 0.88 g (0.11M) of LiH was gradually added thereto. Here, this should be done with care because hydrogen evolves. The reaction solution was stirred, and after confirming no further production of hydrogen, was additionally stirred for another 30 min. Then, 19.7 g (0.10M) of cinnamyl bromide (3-phenylephrine nylallyl bromide) and 3.35 g (0.025M) of LiI were gradually added thereto. The reaction solution was heated to 45 and then stirred vigorously for 12 hours at that temperature. The reaction solution was cooled below 10, and 80 g of ice was first added and 250 ml of water was then added. Thereafter, 25 ml of concentrated HCl was gradually added to maintain the resulting solution at an acidic pH>1. 200 ml of CH₂Cl₂ was added to dissolve the reaction mixture which was then shaken vigorously to separate two layers. The aqueous layer was discarded, and a CH₂Cl₂ layer was treated with an aqueous 2N NaOH solution (100 ml×2) to separate the aqueous layer twice. At this time, the remaining CH₂Cl₂ layer after extraction with an aqueous 2N NaOH solution was used again in Example 8. The thus-separated aqueous solutions were combined and adjusted to an acidic pH>2 using concentrated HCl, thereby forming solids. The resulting solids were filtered and separated to give a Lapachol derivative. The thus-obtained Lapachol derivative was recrystallized from 75% EtOH. The resulting Lapachol derivative was mixed with 50 ml of sulfuric acid, and the mixture was vigorously stirred at room temperature for 10 min and 150 g of ice was added thereto to complete the reaction. 60 ml of CH₂Cl₂ was added to the reaction materials which were then shaken vigorously. Thereafter, a CH₂Cl₂ layer was separated and washed with 5% NaHCO₃. An aqueous layer was extracted once again using 30 ml of CH₂Cl₂, washed with 5% NaHCO₃ and combined with the previously extracted organic layer. The organic layer was concentrated and purified by chromatography on silica gel to give 2.31 g of pure Compound 7.

¹H-NMR (CDCl₃, δ): 8.09(1H, dd, J=1.2, 7.6 Hz), 7.83 (1H, d, J=7.6 Hz), 7.64 (1H, dt, J=1.2, 7.6 Hz), 7.52 (1H, dt, J=1.2, 7.6 Hz), 7.41 (5H, m), 5.27 (1H, dd, J=2.5, 6.0 Hz, 2.77 (1H, m) 2.61 (1H, m), 2.34 (1H, m), 2.08 (1H, m), 0.87 (1H, m)

### Example 8

### Synthesis of Compound 8

The remaining CH₂Cl₂ layer, after extraction with an aqueous 2N NaOH solution in Example 7, was concentrated by distillation under reduced pressure. The resulting concentrates were dissolved in 30 ml of xylene, followed by reflux for 10 hours to induce Claisen Rearrangement. Xylene was concentrated by distillation under reduced pressure and was then mixed with 15 ml of sulfuric acid, without further purification. The resulting mixture was stirred vigorously at room temperature for 10 min and 100 g of ice was added thereto to complete the reaction. 50 ml of CH₂Cl₂ was added to the reaction materials which were shaken vigorously. Thereafter, a CH₂Cl₂ layer was separated and washed with 5% NaHCO₃. An aqueous layer was extracted once again using 20 ml of CH₂Cl₂, washed with 5% NaHCO₃ and combined with the previously extracted organic layer. The organic layer was dried over MgSO₄, concentrated and purified by chromatography on silica gel to give 1.26 g of pure Compound 8.

¹H-NMR (CDCl₃, δ): 8.12 (1H, dd, J=0.8, 8.0 Hz), 7.74 (1H, dd, J=1.2, 7.6 Hz), 7.70 (1H, dt, J=1.2, 7.6 Hz), 7.62 (1H, dt, J=1.6, 7.6 Hz), 7.27 (3H, m), 7.10 (2H, td, J=1.2, 6.4 Hz), 5.38 (1H, qd, J=6.4, 9.2 Hz), 4.61 (1H, d, J=9.2 Hz), 1.17 (3H, d, J=6.4 Hz)

### Example 9

### Synthesis of Compound 9

3.4 g (22 mM) of 1,8-diazabicyclo[5.4.0]undec-7-ene and 1.26 g (15 mM) of 2-methyl-3-butyn-2-ol were dissolved in 10 ml of acetonitrile and the resulting solution was cooled to 0. 3.2 g (15 mM) of trifluoroacetic anhydride was gradually added with stirring to the reaction solution which was then continued to be stirred at 0. 1.74 g (10 mM) of 2-hydroxy-1,4-naphthoquinone and 135 mg (1.0 mM) of cupric chloride (CuCl₂) were dissolved in 10 ml of acetonitrile in another flask, and were stirred. The previously purified solution was gradually added to the reaction solution which was then refluxed for 20 hours. The reaction solution was concentrated by distillation under reduced pressure and was then purified by chromatography on silica gel to give 0.22 g of pure Compound 9.

¹H-NMR (CDCl₃, δ): 8.11 (1H, dd, J=1.2, 7.6 Hz), 7.73 (1H, dd, J=1.2, 7.6 Hz), 7.69 (1H, dt, J=1.2, 7.6 Hz), 7.60 (1H, dt, J=1.6, 7.6 Hz), 4.95 (1H, d, J=3.2 Hz), 4.52 (1H, d, J=3.2 Hz), 1.56 (6H, s)

### Example 10

### Synthesis of Compound 10

0.12 g of Compound 9 was dissolved in 5 ml of MeOH, 10 mg of 5% Pd/C was added thereto, followed by vigorous stirring at room temperature for 3 hours. The reaction solution was filtered through silica gel to remove 5% Pd/C and was concentrated by distillation under reduced pressure to give Compound 10.

¹H-NMR (CDCl₃, δ): 8.05 (1H, td, J=1.2, 7.6 Hz), 7.64 (2H, m), 7.54 (1H, m), 3.48 (3H, s), 1.64 (3H, s), 1.42 (3H, s), 1.29 (3H, s)

### Example 11

### Synthesis of Compound 11

1.21 g (50 mM) of β-Lapachone (Compound 1) and 1.14 g (50 mM) of DDQ (2,3-dichloro-5,6-dicyano-1,4-benzoqinone) were dissolved in 50 ml of carbon tetrachloride and refluxed for 72 hours. The reaction solution was concentrated by distillation under reduced pressure and was then purified by chromatography on silica gel to give 1.18 g of pure Compound 11.

¹H-NMR (CDCl₃, δ): 8.08 (1H, dd, J=1.2, 7.6 Hz), 7.85 (1H, dd, J=0.8, 7.6 Hz), 7.68 (1H, dt, J=1.2, 7.6 Hz), 7.55 (1H, dt, J=1.2, 7.6 Hz), 6.63 (1H, d, J=10.0 Hz), 5.56 (1H, d, J=10.0 Hz), 1.57 (6H, s)

### Example 12

### Synthesis of Compound 12

1.74 g (10 mM) of 2-hydroxy-1,4-naphthoquinone, 3.4 g (50 mM) of 2-methyl-1,3-butadiene (Isoprene), 3.0 g (100 mM) of paraformaldehyde and 20 ml of 1,4-dioxane were placed into a pressure vessel, and were heated with stirring at 100 for 48 hours. The reaction vessel was cooled to room temperature, and contents therein were filtered. The filtrate was concentrated by distillation under reduced pressure and was then purified by chromatography on silica gel to give 238 mg of Compound 12, as a 2-vinyl derivative of β-Lapachone.

¹H-NMR (CDCl₃, δ): 8.07 (1H, dd, J=1.2, 7.6 Hz), 7.88 (1H, dd, J=0.8, 7.6 Hz), 7.66 (1H, dt, J=1.2, 7.6 Hz), 7.52 (1H, dt, J=0.8, 7.6 Hz), 5.87 (1H, dd, J=10.8, 17.2 Hz), 5.18 (1H, d, J=10.8 Hz), 5.17 (1H, 17.2 Hz), 2.62 (1H, m), 2.38 (1H, m), 2.17 (3H, s), 2.00 (1H, m), 1.84 (1H, m)

### Example 13

### Synthesis of Compound 13

1.74 g (10 mM) of 2-hydroxy-1,4-naphthoquinone, 4.8 g (50 mM) of 2,4-dimethyl-1,3-pentadiene and 3.0 g (100 mM) of paraformaldehyde were dissolved in 20 ml of 1,4-dioxane, and the resulting mixture was refluxed with vigorous stirring for 10 hours. The reaction vessel was cooled to room temperature, and contents therein were filtered to remove paraformaldehyde from solids. The filtrate was concentrated by distillation under reduced pressure and was then purified by chromatography on silica gel to give 428 mg of Compound 13, as a β-Lapachone derivative.

¹H-NMR (CDCl₃, δ): 8.06 (1H, dd,J=1.2, 7.6 Hz), 7.83 (1H, dd, J=0.8, 7.6 Hz), 7.65 (1H, dt, J=1.2, 7.6 Hz), 7.50 (1H, dt, J=0.8, 7.6 Hz), 5.22 (1H, bs), 2.61 (1H, m), 2.48 (1H, m), 2.04 (1H, m), 1.80 (3H, d, J=1.0 Hz), 1.75 (1H, m), 1.72 (1H, d, J=1.0 Hz), 1.64 (3H, s)

### Example 14

### Synthesis of Compound 14

5.3 g (30 mM) of 2-hydroxy-1,4-naphthoquinone, 20.4 g (150 mM) of 2,6-dimethyl-2,4,6-octatriene and 9.0 g (300 mM) of paraformaldehyde were dissolved in 50 ml of 1,4-dioxane, and the resulting mixture was refluxed with vigorous stirring for 10 hours. The reaction vessel was cooled to room temperature, and contents therein were filtered to remove paraformaldehyde from solids. The filtrate was concentrated by distillation under reduced pressure and was then purified by chromatography on silica gel to give 1.18 g of Compound 14, as a β-Lapachone derivative.

¹H-NMR (CDCl₃, δ): 8.07 (1H, dd, J=1.2, 7.6 Hz), 7.87 (1H, dd, J=0.8, 7.6 Hz), 7.66 (1H, dt, J=1.2, 7.6 Hz), 7.51 (1H, dt, J=0.8, 7.6 Hz), 6.37 (1H, dd, J=11.2, 15.2 Hz), 5.80 (1H, broad d, J=11.2 Hz), 5.59 (1H, d, J=15.2 Hz), 2.67 (1H, dd, J=4.8, 17.2 Hz), 2.10 (1H, dd, J=6.0, 17.2 Hz), 1.97 (1H, m), 1.75 (3H, bs), 1.64 (3H, bs), 1.63 (3H, s), 1.08 (3H, d, J=6.8 Hz)

### Example 15

### Synthesis of Compound 15

5.3 g (30 mM) of 2-hydroxy-1,4-naphthoquinone, 20.4 g (50 mM) of terpinen and 9.0 g (300 mM) of paraformaldehyde were dissolved in 50 ml of 1,4-dioxane, and the resulting mixture was refluxed with vigorous stirring for 10 hours. The reaction vessel was cooled to room temperature, and contents therein were filtered to remove paraformaldehyde from solids. The filtrate was concentrated by distillation under reduced pressure and was then purified by chromatography on silica gel to give 1.12 g of Compound 15, as a tetracyclic o-quinone derivative.

¹H-NMR (CDCl₃, δ): 8.06 (1H, d, J=7.6 Hz), 7.85 (1H, d, J=7.6 Hz), 7.65 (1H, t, J=7.6 Hz), 7.51 (1H, t, J=7.6 Hz), 5.48 (1H, broad s), 4.60 (1H, broad s), 2.45 (1H, d, J=16.8 Hz), 2.21 (1H, m), 2.20 (1H, d, J=16.8 Hz), 2.09 (1H, m), 1.77 (1H, m), 1.57 (1H, m), 1.07 (3H, s), 1.03 (3H, d, J=0.8 Hz), 1.01 (3H, d, J=0.8 Hz), 0.96 (1H, m)

### Example 16

### Synthesis of Compounds 16 and 17

17.4 g (0.10M) of 2-hydroxy-1,4-naphthoquinone was dissolved in 120 ml of DMSO, and 0.88 g (0.11M) of LiH was gradually added thereto. Here, this should be done with care because hydrogen evolves. The reaction solution was stirred, and after confirming no further production of hydrogen, was additionally stirred for another 30 min. Then, 16.3 g (0.12M) of crotyl bromide and 3.35 g (0.025M) of LiI were gradually added thereto. The reaction solution was heated to 45 and then vigorously stirred for 12 hours at that temperature. The reaction solution was cooled below 10, and 80 g of ice was first added and 250 ml of water was then added. Thereafter, 25 ml of concentrated HCl was gradually added to maintain the resulting solution at an acidic pH>1. 200 ml of CH₂Cl₂ was added to dissolve the reaction mixture which was then shaken vigorously to separate two layers. The aqueous layer was discarded, and a CH₂Cl₂ layer was treated with an aqueous 2N NaOH solution (100 ml×2) to separate the aqueous layer twice. At this time, the remaining CH₂Cl₂ layer after extraction with an aqueous 2N NaOH solution was used in Example 17. The thus-separated aqueous solutions were combined and adjusted to an acidic pH>2 using concentrated HCl, thereby forming solids. The resulting solids were filtered and separated to give a Lapachol derivative. The thus-obtained Lapachol derivative was recrystallized from 75% EtOH. The resulting Lapachol derivative was mixed with 50 ml of sulfuric acid, and the mixture was vigorously stirred at room temperature for 10 min, followed by addition of 150 g of ice to complete the reaction. 60 ml of CH₂Cl₂ was added to the reaction materials which were then shaken vigorously. Thereafter, a CH₂Cl₂ layer was separated and washed with 5% NaHCO₃. An aqueous layer was extracted once again using 30 ml of CH₂Cl₂, washed with 5% NaHCO₃ and combined with the previously extracted organic layer. The organic layer was concentrated and purified by chromatography on silica gel to give 1.78 g and 0.43 g of pure Compounds 16 and 17, respectively.

¹H-NMR (CDCl₃, δ) of Compound 16: δ8.07 (1H, dd, J=0.8, 6.8 Hz), 7.64 (2H, broad d, J=3.6 Hz), 7.57 (1H, m), 5.17 (1H, qd, J=6.0, 8.8 Hz), 3.53 (1H, qd, J=6.8, 8.8 Hz), 1.54 (3H, d, 6.8 Hz), 1.23 (3H, d, 6.8 Hz)

¹H-NMR (CDCl₃, δ) of Compound 17: δ8.06 (1H, d, J=0.8, 7.2 Hz), 7.65 (2H, broad d, J=3.6 Hz), 7.57 (1H, m), 4.71 (1H, quintet, J=6.4 Hz), 3.16 (1H, quintet, J=6.4 Hz), 1.54 (3H, d, 6.4 Hz), 1.38 (3H, d, 6.4 Hz)

### Example 17

### Synthesis of Compounds 18 and 19

The remaining CH₂Cl₂ layer, after extraction with an aqueous 2N NaOH solution in Example 16, was concentrated by distillation under reduced pressure. The resulting concentrates were dissolved in 30 ml of xylene, followed by reflux for 10 hours to induce Claisen Rearrangement. Xylene was concentrated by distillation under reduced pressure and was then mixed with 15 ml of sulfuric acid, without further purification. The resulting mixture was stirred vigorously at room temperature for 10 min and 100 g of ice was added thereto to complete the reaction. 50 ml of CH₂Cl₂ was added to the reaction materials which were shaken vigorously. Thereafter, a CH₂Cl₂ layer was separated and washed with 5% NaHCO₃. An aqueous layer was extracted once again using 20 ml of CH₂Cl₂, washed with 5% NaHCO₃ and combined with the previously extracted organic layer. The organic layer was dried over MgSO₄, concentrated and purified by chromatography on silica gel to give 0.62 g and 0.43 g of pure Compounds 18 and 19, respectively.

¹H-NMR (CDCl₃, δ) of Compound 18: 8.06 (1H, dd, J=0.8, 7.2 Hz), 7.81 (1H, dd, J=0.8, 7.6 Hz), 7.65 (1H, dt, J=0.8, 7.6 Hz), 7.51 (1H, dt, J=0.8, 7.2 Hz), 4.40 (1H, m), 2.71 (1H, m), 2.46 (1H, m), 2.11 (1H, m), 1.71 (1H, m), 1.54 (3H, d, 6.4 Hz), 1.52 (1H, m)

¹H-NMR (CDCl₃, δ) of Compound 19: 8.08 (1H, d, J=0.8, 7.2 Hz), 7.66 (2H, broad d, J=4.0 Hz), 7.58 (1H, m), 5.08 (1H, m), 3.23 (1H, dd, J=9.6, 15.2 Hz), 2.80 (1H, dd, J=7.2, 15.2 Hz), 1.92 (1H, m), 1.82 (1H, m), 1.09 (3H, t, 7.6 Hz)

### Example 18

### Synthesis of Compound 20

17.4 g (0.10M) of 2-hydroxy-1,4-naphthoquinone was dissolved in 120 ml of DMSO, and 0.88 g (0.11M) of LiH was gradually added thereto. Here, this should be done with care because hydrogen evolves. The reaction solution was stirred, and after confirming no further production of hydrogen, was additionally stirred for another 30 min. Then, 21.8 g (0.10M) of geranyl bromide and 3.35 g (0.025M) of LiI were gradually added thereto. The reaction solution was heated to 45° C. and then vigorously stirred for 12 hours at that temperature. The reaction solution was cooled below 10° C., and 80 g of ice was first added and 250 ml of water was then added. Thereafter, 25 ml of concentrated HCl was gradually added to maintain the resulting solution at an acidic pH>1. 200 ml of CH₂Cl₂ was added to dissolve the reaction mixture which was then shaken vigorously to separate two layers. The aqueous layer was discarded, and a CH₂Cl₂ layer was treated with an aqueous 2N NaOH solution (100 ml×2) to separate the aqueous layer twice. The thus-separated aqueous solutions were combined and adjusted to an acidic pH>2 using concentrated HCl, thereby forming solids. The resulting solids were filtered and separated to give 2-geranyl-3-hydroxy-1,4-naphthoquinone. The thus-obtained product was mixed with 50 ml of sulfuric acid without further purification, and the mixture was vigorously stirred at room temperature for 10 min, followed by addition of 150 g of ice to complete the reaction. 60 ml of CH₂Cl₂ was added to the reaction materials which were then shaken vigorously. Thereafter, a CH₂Cl₂ layer was separated and washed with 5% NaHCO₃. An aqueous layer was extracted once again using 30 ml of CH₂Cl₂, washed with 5% NaHCO₃ and combined with the previously extracted organic layer. The organic layer was concentrated and purified by chromatography on silica gel to give 3.62 g of pure Compound 20.

¹H-NMR (CDCl₃, δ): 8.05 (1H, d, J=7.6 Hz), 7.77 (1H, d, J=7.6 Hz), 7.63 (1H, t, J=7.6 Hz), 7.49 (1H, t, J=7.6 Hz), 2.71 (1H, dd, J=6.0, 17.2 Hz), 2.19 (1H, dd, J=12.8, 17.2 Hz), 2.13 (1H, m), 1.73 (2H, m), 1.63 (1H, dd, J=6.0, 12.8 Hz), 1.59 (1H, m), 1.57 (1H, m), 1.52 (1H, m), 1.33 (3H, s), 1.04 (3H, s), 0.93 (3H, s)

### Example 19

### Synthesis of Compound 21

Compound 21 was obtained in the same manner as in Example 1, except that 6-chloro-2-hydroxy-1,4-naphthoquinone was used instead of 2-hydroxy-1,4-naphthoquinone.

¹H-NMR (CDCl₃, δ): 8.02 (1H, d, J=8 Hz), 7.77 (1H, d, J=2 Hz), 7.50 (1H, dd, J=2, 8 Hz), 2.60 (2H, t, J=7 Hz), 1.87(2H, t, J=7 Hz) 1.53 (6H, s)

### Example 20

### Synthesis of Compound 22

Compound 22 was obtained in the same manner as in Example 1, except that 2-hydroxy-6-methyl-1,4-naphthoquinone was used instead of 2-hydroxy-1,4-naphthoquinone.

¹-NMR (CDCl₃, δ): 7.98 (1H, d, J=8 Hz), 7.61 (1H, d, J=2 Hz), 7.31 (1H, dd, J=2, 8 Hz), 2.58 (2H, t, J=7 Hz), 1.84(2H, t, J=7 Hz) 1.48 (6H, s)

### Example 21

### Synthesis of Compound 23

Compound 23 was obtained in the same manner as in Example 1, except that 6,7-dimethoxy-2-hydroxy-1,4-naphthoquinone was used instead of 2-hydroxy-1,4-naphthoquinone.

¹H-NMR (CDCl₃, δ): 7.56 (1H, s), 7.25 (1H, s), 3.98 (6H, s), 2.53 (2H, t, J=7 Hz), 1.83(2H, t, J=7 Hz) 1.48 (6H, s)

### Example 22

### Synthesis of Compound 24

Compound 24 was obtained in the same manner as in Example 1, except that 1-bromo-3-methyl-2-pentene was used instead of 1-bromo-3-methyl-2-butene.

¹H-NMR (CDCl₃, δ): 7.30˜8.15 (4H, m), 2.55 (2H, t, J=7 Hz), 1.83(2H, t, J=7 Hz), 1.80(2H, q, 7 Hz) 1.40 (3H, s), 1.03(3H, t, J=7 Hz)

### Example 23

### Synthesis of Compound 25

Compound 25 was obtained in the same manner as in Example 1, except that 1-bromo-3-ethyl-2-pentene was used instead of 1-bromo-3-methyl-2-butene.

¹H-NMR (CDCl₃, δ): 7.30˜8.15 (4H, m), 2.53 (2H, t, J=7 Hz), 1.83(2H, t, J=7 Hz), 1.80(4H, q, 7 Hz) 0.97(6H, t, J=7 Hz)

### Example 24

### Synthesis of Compound 26

Compound 26 was obtained in the same manner as in Example 1, except that 1-bromo-3-phenylephrinenyl-2-butene was used instead of 1-bromo-3-methyl-2-butene.

¹H-NMR (CDCl₃, δ): 7.15˜8.15 (9H, m), 1.90˜2.75 (4H, m), 1.77 (3H, s)

### Example 25

### Synthesis of Compound 27

Compound 27 was obtained in the same manner as in Example 1, except that 2-bromo-ethylidenecyclohexane was used instead of 1-bromo-3-methyl-2-butene.

¹H-NMR (CDCl₃, δ): 7.30˜8.25 (4H, m), 2.59 (2H, t, J=7 Hz), 1.3˜2.15 (12H, m)

### Example 26

### Synthesis of Compound 28

Compound 28 was obtained in the same manner as in Example 1, except that 2-bromo-ethylidenecyclopentane was used instead of 1-bromo-3-methyl-2-butene.

¹H-NMR (CDCl₃, δ): 7.28˜8.20 (4H, m), 2.59 (2H, t, J=7 Hz), 1.40˜2.20 (10H, m)

### Example 27

### Synthesis of Compound 29

8.58 g (20 mM) of Compound 5 synthesized in Example 5 was dissolved in 1000 ml of carbon tetrachloride, followed by addition of 11.4 g (50 mM) of 2,3-dichloro-5,6-dicyano-1,4-benzoqinone, and the resulting mixture was refluxed for 96 hours. The reaction solution was concentrated by distillation under reduced pressure and the resulting red solids were then recrystallized from isopropanol, thereby obtaining 7.18 g of pure Compound 29.

¹H-NMR (CDCl₃, δ): 8.05 (1H, dd, J=1.2, 7.6 Hz), 7.66 (1H, dd, J=1.2, 7.6 Hz), 7.62 (1H, dt, J=1.2, 7.6 Hz), 7.42 (1H, dt, J=1.2, 7.6 Hz), 6.45 (1H, q, J=1.2 Hz), 2.43 (3H, d, J=1.2 Hz)

### Example 28

### Synthesis of Compound 30

Analogous to a synthesis method as taught in J. Org. Chem., 55 (1990) 4995-5008, 4,5-dihydro-3-methylbenzo[1,2-b]furan-4,5-dione {Benzofuran-4,5-dione} was synthesized using p-benzoquinone and 1-(N-morpholine)propene. 1.5 g (9.3 mM) of the thus-prepared benzofuran-4,5-dione and 3.15 g (28.2 mM) of 1-acetoxy-1,3-butadiene were dissolved in 200 ml of benzene, and the resulting mixture was refluxed for 12 hours. The reaction solution was cooled to room temperature and concentrated by distillation under reduced pressure. This was followed by chromatography on silica gel to give 1.13 g of pure Compound 30.

¹H-NMR (CDCl₃, δ): 8.05 (1H, dd, J=1.2, 7.6 Hz), 7.68 (1H, dd, J=1.2, 7.6 Hz), 7.64 (1H, td, J=1.2, 7.6 Hz), 7.43 (1H, td, J=1.2, 7.6 Hz), 7.26 (1H, q, J=1.2 Hz), 2.28 (3H, d, J=1.2 Hz)

### Example 29

### Synthesis of Compounds 31 and 32

1.5 g (9.3 mM) of 4,5-dihydro-3-methylbenzo[1,2-b]furan-4,5-dione {Benzofuran-4,5-dione} and 45 g (0.6M) of 2-methyl-1,3-butadiene were dissolved in 200 ml of benzene, and the resulting mixture was refluxed for 5 hours. The reaction solution was cooled to room temperature and completely concentrated by distillation under reduced pressure. The thus-obtained concentrates were dissolved again in 150 ml of carbon tetrachloride, followed by addition of 2.3 g (10 mM) of 2,3-dichloro-5,6-dicyano-1,4-benzoqinone, and the resulting mixture was further refluxed for 15 hours. The reaction solution was cooled and concentrated by distillation under reduced pressure. The resulting concentrates were purified by chromatography on silica gel to give 0.13 g and 0.11 g of pure Compounds 31 and 32, respectively.

¹H-NMR (CDCl₃, δ) of Compound 31: 7.86 (1H, s), 7.57 (1H, d, J=8.1 Hz), 7.42 (1H, d, J=8.1 Hz), 7.21 (1H, q, J=1.2 Hz), 2.40 (3H, s), 2.28 (1H, d, J=1.2 Hz)

¹H-NMR (CDCl₃, δ) of Compound 32: δ7.96 (1H, d, J=8.0 Hz), 7.48 (1H, s), 7.23 (2H, m), 2.46 (3H, s), 2.28 (1H, d, J=1.2 Hz)

### Methods and Materials

### 1. Selection and Assignment of Experimental Subjects

Experimental animals were divided into three groups:

Group 1: Non-treated normal group (n=6),

Group 2: Regular chow-fed control group (n=7) as a glaucoma model, and

Group 3: Experimental group (n=7) fed with a rodent chow containing 50 mg/kg of a pharmaceutical composition including compound 1 of experimental example 1 as an active ingredient, as a glaucoma model.

### 2. Glaucoma Experimental Model—Establishment of Optic Nerve Injury Model by Transpupillary Thermotherapy (TTT) Laser Treatment

8-week-old C57BL/6 mice were anesthetized with an intraperitoneal injection of a mixture of ketamine (100 mg/kg) and xylazine (5 mg/kg), and a mydriatic agent was applied to dilate the pupils of the eyes. Thereafter, an application of transpupillary thermotherapy (TTT), 200 spot size, 50-mW power, and 30-sec duration, was performed over the optic disc of an eye. The aiming beam of the laser was focused on the centre of the optic disc, a viscoelastic material was instilled, a cover glass was placed, and laser beams were irradiated while confirming the optic disc through the dilated pupils by naked eyes. The thus-established optic nerve injury model will be hereinafter referred to as a TTT laser model.

### 3. Pair Feeding

In order to confirm changes in body weight of experimental animals, mice having similar body weight were selected and paired from the experimental group and the control group. One day after laser treatment of a TTT laser model, mice were fed for 2 weeks according to the pair-feeding method. 24 hours after feeding of the experimental group, feeding of the control group was initiated. The control group was fed the same amount of a regular chow (solid chow: 5053, Labdiet) as compound 1 of experimental example 1 that was given to the experimental group on the previous day.

### 4. Statistical Analysis

Viability of retinal ganglion cells and axons was analyzed for each group, and the difference between two groups (such as an experiment vs. control group) was determined to be statistically significant when P<0.05.

### Experimental Example 1

### Viability of Retinal Ganglion Cells (RGCs)

### a) Labeling of Retinal Ganglion Cells

On day 13 after laser irradiation of a TTT laser model, animals were anesthetized in the same manner as in the previous TTT laser treatment, followed by exposure of the optic nerve and incision of the optic nerve sheath using an MVR blade. The exposed optic nerve tissue was cut, and DTMR (Dextran TetradiMethyl Rhodamine) crystals were applied to the proximal cut surface of the optic nerve to label the RGCs by axonal transport.

### b) Viability Assay for Retinal Ganglion Cells

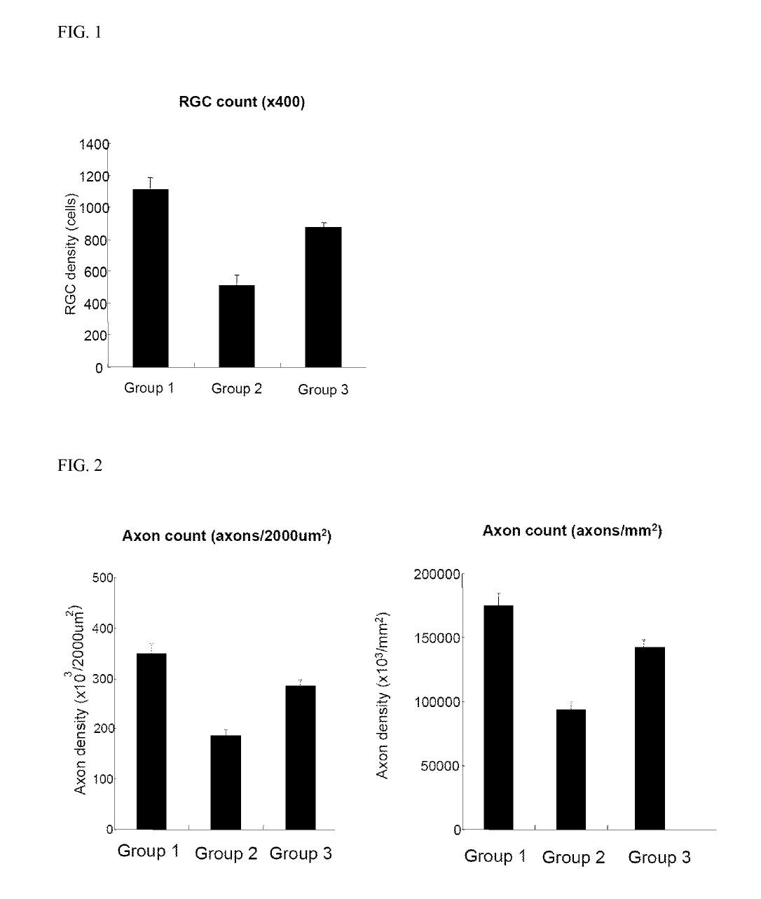

Twenty-four hours after labeling, the animals were euthanized and the eyes were enucleated and fixed for 2 hours with neutral formalin. Then, the cornea and the crystalline lens were removed from the corneal limbus, and the retina was separated from the choroid. The retina was dissected and flat mounted on a slide. Four radial cuts were made around the optic disk, followed by addition of an aqueous mountant. Under a fluorescence microscope (×400), the fluorescently labeled retinal ganglion cells were counted in 12 regions in the four quadrants of each retina approximately 0.5 mm, 1 mm and 1.5 mm from the edge of the optic disc. The counting was performed by three observers in a masked fashion and averaged. The results obtained are shown in FIG. 1.

Referring to FIG. 1, it can be seen that the TTT control group (Group 2), i.e. an animal group of the TTT laser model which was laser-irradiated and fed with a regular diet, exhibited a significant decrease in a density of retinal ganglion cells, corresponding to a 2/1 level of an animal group (Group 1) with a normal density of retinal ganglion cells. However, an animal group (Group 3) with administration of the pharmaceutical composition (compound 1 of experimental example 1) in accordance with the present invention exhibited a significant increase in a density of retinal ganglion cells, 1.7-fold or higher than the TTT control group (Group 2), thus confirming that the cellular damage was delayed and the damaged cells returned to normal conditions.

From these results, it can be seen that the pharmaceutical composition in accordance with the present invention can be used as a novel therapeutic agent for glaucoma that arises due to glaucomatous damage of retinal ganglion cells (RGCs) resulting in blockage of information communication.

### Experimental Example 2

### Axonal Viability

### a) Preparation of Tissue Sections and Light Microscopic Examination

On day 14 after laser irradiation of a TTT laser model, three animals per group were anesthetized with a mixture of ketamine and xylazine, and the eyes were enucleated and fixed in neutral formalin. The tissue sections, prepared following the dehydration and paraffinization processes, were stained with hematoxylin and eosin (H&E) to compare the degree of damage of the retinal tissue and the retinal thickness between individual animal groups. The retinal cross-section and the optic nerve cross-section were subjected to special staining to thereby compare the degree of damage of the optic nerve fiber and the axonal viability between animal groups.

### b) Bodian Staining and Axonal Viability Assay

The retinal cross-section and the optic nerve cross-section were treated with a silver solution for 48 hours, and color development was carried out using a reducing agent, followed by toning and fixation. Then, the degree of damage of the optic nerve fiber was examined under a light microscope. In order to evaluate the axonal viability, the stained axons were counted with a light microscope (×1000), in 20 regions at intervals of 10 in the four quadrants of each retina, from the center of the optic nerve tissue section slide. The counting was performed by three observers in a masked fashion and averaged. The results obtained are shown in FIG. 2.

Referring to FIG. 2, it can be confirmed that the TTT control group (Group 2) exhibited a significant decrease in the axonal density due to TTT laser irradiation, that is, a 2/1 level of a normal group (Group 1), whereas an animal group (Group 3) with administration of the pharmaceutical composition (compound 1 of experimental example 1) in accordance with the present invention exhibited a significant increase in the axonal density, 1.5-fold or higher than the TTT control group (Group 2).

Therefore, the pharmaceutical composition in accordance with the present invention can be effectively used for the treatment and prevention of glaucoma which is a group of diseases occurring as a result of progressive loss of axons of the retinal nerve fiber.

### Experimental Example 3

### Effects of Inventive Composition on Body Weight of Mice

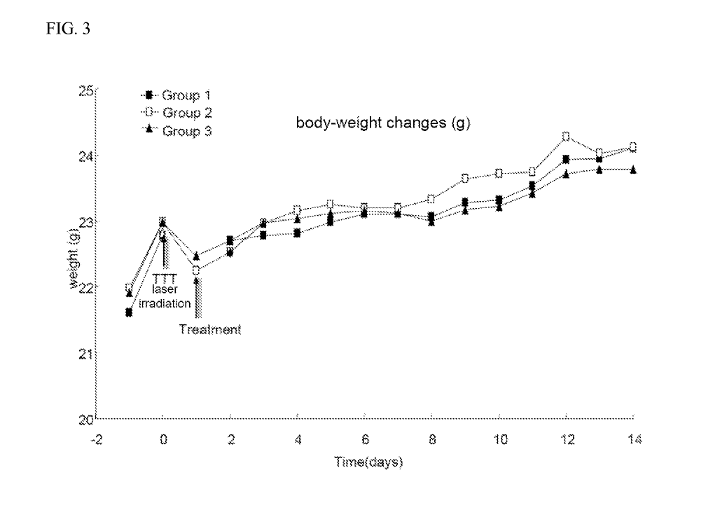

In order to confirm whether administration of a pharmaceutical composition has effects on body weight of mice in an experimental animal group which was given a pharmaceutical composition (compound 1 of experimental example 1) in accordance with the present invention, mice were fed for 2 weeks according to the pair-feeding method, after laser treatment of a TTT laser model. Measurement results of body weight in individual animal groups are shown in FIG. 3.

Referring to FIG. 3, administration of the pharmaceutical composition in accordance with the present invention exhibited feeding versus weight gain profiles similar to a normal group while exhibiting significant prophylactic and therapeutic effects against glaucoma, thus maintaining dietary intake behavior and metabolic activity similar to the normal group. From these results, the pharmaceutical composition in accordance with the present invention does not appear to cause significant adverse effects such as hypometabolism, and is expected to be effective as a pharmaceutical composition for the treatment and prevention of glaucoma.

### INDUSTRIAL APPLICABILITY

As apparent from the foregoing, a pharmaceutical composition in accordance with the present invention prevents the degeneration of retinal ganglion cells (RGCs) and RGC axons forming the optic nerve and facilitates the recovery of the damaged RGCs and axons to thereby have excellent effects on the treatment and prevention of glaucoma.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A pharmaceutical composition for the treatment and prevention of glaucoma, comprising: (a) a therapeutically effective amount of a compound represented by Formula 1: 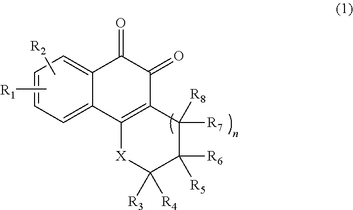
wherein:
R₁ and R₂ are each independently hydrogen, halogen, hydroxyl, or C₁-C₆ lower alkyl or alkoxy, or R₁ and R₂ may be taken together to form a cyclic structure which may be saturated or partially or completely unsaturated;
R₃, R₄, R₅, R₆, R₇ and R₈ are each independently hydrogen, hydroxyl, C₁-C₂₀ alkyl, alkene or alkoxy, cycloalkyl, heterocycloalkyl, aryl or heteroaryl, or two of R₃ to R₈ may be taken together to form a cyclic structure which may be saturated or partially or completely unsaturated;
X is selected from the group consisting of C(R)(R′), N(R″) wherein R, R′ and R″ are each independently hydrogen or C₁-C₆ lower alkyl, O and S; and
n is 0 or 1, with proviso that when n is 0, carbon atoms adjacent to n form a cyclic structure via a direct bond.

2. The composition according to claim 1, wherein X is O.

3. The composition according to claim 1, wherein the prodrug is a compound represented by Formula 1a below: 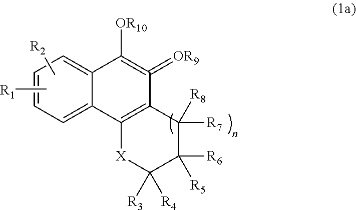
wherein,
R₁, R₂, R₃, R₄, R₅, R₆, R₇, R₈, X and n are as defined in Formula 1;
R₉ and R₁₀ are each independently —SO₃—Na⁺ or substituent represented by Formula A below or a salt thereof, 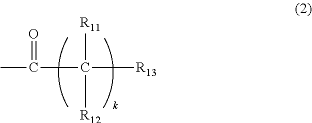
wherein,
R₁₁ and R₁₂ are each independently hydrogen or substituted or unsubstituted C₁-C₂₀ linear alkyl or C₁-C₂₀ branched alkyl,
R₁₃ is selected from the group consisting of substituents i) to viii) below,
i) hydrogen;
ii) substituted or unsubstituted C₁-C₂₀ linear alkyl or C₁-C₂₀ branched alkyl;
iii) substituted or unsubstituted amine;
iv) substituted or unsubstituted C₃-C₁₀ cycloalkyl or C₃-C₁₀ heterocycloalkyl;
v) substituted or unsubstituted C₄-C₁₀ aryl or C₄-C₁₀ heteroaryl;
vi) —(CRR′—NR″CO)₁—R₁₄, wherein R, R′ and R″ are each independently hydrogen or substituted or unsubstituted C₁-C₂₀ linear alkyl or C₁-C₂₀ branched alkyl, R₁₄ is selected from the group consisting of hydrogen, substituted or unsubstituted amine, cycloalkyl, heterocycloalkyl, aryl and heteroaryl, 1 is selected from the 1˜5;
vii) substituted or unsubstituted carboxyl;
viii) —OSO₃—Na⁺;
k is selected from the 0˜20, with proviso that when k is 0, R₁₁ and R₁₂ are not anything, and R₁₃ is directly bond to a carbonyl group.

4. The composition according to claim 1, wherein the compound of Formula 1 is selected from compounds of Formulas 3 and 4 below: 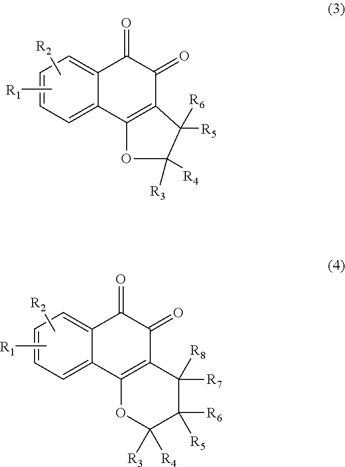
wherein R₁, R₂, R₃, R₄, R₅, R₆, R₇ and R₈ are as defined in Formula 1.

5. The composition according to claim 1, wherein each of R₁ and R₂ is respectively hydrogen.

6. The composition according to claim 4, wherein the compound of Formula 3 is a compound of Formula 3a below in which R₁, R₂ and R₄ are respectively hydrogen, or a compound of Formula 3b below in which R₁, R₂ and R₆ are respectively hydrogen:

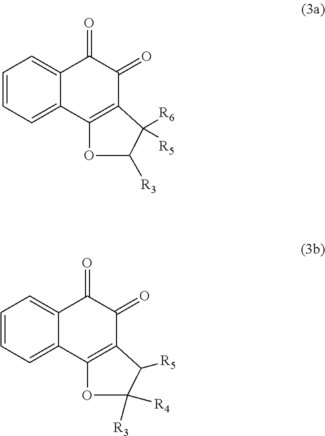

7. The composition according to claim 4, wherein the compound of Formula 4 is a compound of Formula 4a below in which R₁, R₂, R₅, R₆, R₇ and R₈ are respectively hydrogen:

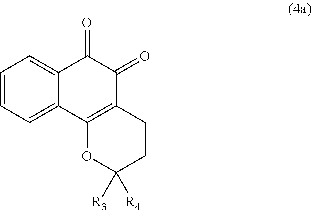

8. The composition according to claim 1, wherein the compound of Formula 1 is contained in an amorphous structure.

9. The composition according to claim 8, wherein the amorphous structure is obtained in formulating the compound of Formula 1, as an active component, or the pharmaceutical composition containing the compound to the form of a fine particle.

10. The composition according to claim 9, wherein the formulation for form of a fine particle is carried out by using the particle micronization method selected from the group consisting of mechanical milling, spray drying, precipitation method, homogenization, and supercritical micronization.

11. The composition according to claim 1, wherein the pharmaceutical composition is prepared into an intestine-targeted formulation.

12. The composition according to claim 15, wherein the intestine-targeted formulation is carried out by addition of a pH sensitive polymer.

13. The composition according to claim 11, wherein the intestine-targeted formulation is carried out by addition of a biodegradable polymer which is decomposable by an intestine-specific bacterial enzyme.

14. The composition according to claim 11, wherein the intestine-targeted formulation is carried out by addition of a biodegradable matrix which is decomposable by an intestine-specific bacterial enzyme.

15. The composition according to claim 11, wherein the intestine-targeted formulation is carried out by a configuration with time-course release of the drug after a lag time (‘time-specific delayed-release formulation’).

16. A method for preparing a medicine for the treatment and/or prevention of glaucoma using the compound of Formula 1 according to claim 1.
